(19) United States
(12) Patent Application Publication
(10) Pub. No.: US 2026/0272677 A1
(43) Pub. Date: Sep. 17, 2026

(54) Angled Plate Inserter

(71) Applicant: VB Spine LLC, Leesburg, VA (US)

(72) Inventor: Liam Patrick Barnes, Round Hill, VA (US)

(73) Assignee: VB Spine LLC, Leesburg, VA (US)

(21) Appl. No.: 19/563,439

(22) Filed: Mar. 11, 2026

Related U.S. Application Data
(60) Provisional application No. 63/770,675, filed on Mar. 12, 2025.

(51) Int. Cl. A61F 2/46; A61F 2/44

(52) U.S. Cl. CPC A61F 2/4611; A61F 2/4455; A61F 2002/4627; A61F 2002/4628; A61F 2002/4629

(57) ABSTRACT

A plate insertion apparatus includes a shaft, a distal tip, a driver, and an actuator. The shaft has a proximal end and a distal end and extends along a first axis. The distal tip extends from the distal end of the shaft along a second axis. The distal tip includes a first claw, a second claw, and a space between the first and second claws. The second axis is different than the first axis. The driver is positioned within the space of the distal tip and is biased by a spring extending along the second axis. The actuator is adjacent to the proximal end, wherein actuation of the actuator in a first direction causes the first and second claws to expand and actuation of the actuator in a second direction causes the first and second claws to contract.

### CROSS-REFERENCE TO RELATED APPLICATIONS

This application claims the benefit of the filing date of United States Provisional Patent Application No. 63/770,675 filed Mar. 12, 2025, the disclosure of which is hereby incorporated herein by reference in its entirety.

### BACKGROUND OF THE INVENTION

The human spine and the associated nervous system are integral parts of human anatomy. When something is wrong with the spine it can cause extensive issues including pain, discomfort, limited range of movement, and other negative effects. These problems can result from a plethora of causes, including disc degeneration, trauma, and disease. Spinal surgery has become a common solution to address these issues in patients.

A common spinal surgical procedure is one in which a disc of the spine is removed and replaced with a prosthetic implant to fuse together adjacent vertebrae. This sometimes involves the fixation of the implant to the vertebrae with an intermediate plate. For instance, the CAYMAN ® plate and CASCADIA ® Lateral implant, both of which are offered by VB Spine of Leesburg, VA, are often utilized together in a spinal fusion procedure.

However, even these surgeries are not without their own complications. Spinal operations require accuracy and precision utilizing instruments that need to fit in a limited area. An increased risk of complications can arise the more instruments are utilized in the procedure as well as the overall time the procedure takes. This is particularly evident when you are marrying plates with implants in the rather small confines of the spine.

Therefore, there exists a need for improved instrumentation for use in such procedures, including an improved plate inserter.

### BRIEF SUMMARY OF THE INVENTION

The various disclosed embodiments include a system and method for an angled plate inserter and insertion of a plate into a patient, for instance, in conjunction with an implant. The plate inserter includes a housing extending along a first axis and an angled distal tip extending from a distal end of the housing along a second axis different from the first axis. The distal tip includes a first claw and a second claw configured to receive at least a plate. In an embodiment, the first and second claws are further configured to receive an interbody device and a fastening member configured to attach the interbody device with the plate. The distance between first and second claws is configured to decrease or increase depending on actuation of an actuator. The actuator is positioned near a proximal end of the housing opposite that of the distal tip. Actuation of the actuator in a first direction causes first and second claws to contract and actuation of the actuator in a second direction causes the claws to expand. Contraction of the claws is configured to secure a plate onto the plate inserter. Whereas expansion of the claws is configured to release a plate from securement on the plate inserter or to prepare the plate inserter to receive a plate thereon. In an embodiment, the distal tip of the plate inserter includes a driver and a compression member. The driver is configured to drive a fastening member as the fastening member advances such that the plate inserter with at least a plate and fastening member inserted thereon is configured to be secured to an interbody device as the plate inserter advances towards the spine. Specifically, the compression member biases the driver such that an inserted fastening member can be driven as the plate inserter advances into a patient.

This is desirable as the plate inserter can both insert a plate and fasten the fastening member to the plate and interbody device during the same insertion, reducing the need for two instruments to perform each task separately or for the same instrument to perform both tasks during two different insertions. The use of two different instruments or the use of the duplication of steps with the same instrument to insert the plate and to insert the fastening member increases the likelihood of patient complications and increases overall surgical time. Specifically, spinal surgeries often involve limited working areas as many approaches are close to critical areas (e.g., organs, nervous system, blood vessels, etc.). The more instruments that are traveling into and out of the tight surgical area increases the risk of contacting a critical area. Furthermore, it is more difficult to insert an interbody device, then insert a plate, and then fasten the plate with the interbody device than to have all two or more of those components received by the distal tip and inserted and fastened simultaneously.

Additionally, the fastening member can be depressed against the compression member within the distal tip of the plate inserter when desirable. As a non-limiting example, the fastening member can be depressed within the plate inserter until the fastening member is aligned with a pre-inserted interbody, such that when aligned, the plate inserter is actuated from the depressed state to an actuated state in which the compression member biases the fastening member against the plate and into a receiving member (e.g., threads) of the interbody.

In accordance with an aspect of the present disclosure, a plate insertion apparatus includes a shaft, a distal tip, and an actuator. The shaft extends along a first axis and has a proximal end and a distal end. The distal tip extends from the distal end of the shaft along a second axis. The distal tip includes a first claw, a second claw, and a cavity between the first and second claws. The second axis is different from the first axis. The actuator is adjacent to the proximal end, wherein actuation of the actuator in a first direction causes the first and second claws to expand and actuation of the actuator in a second direction causes the first and second claws to contract.

In other embodiments of this aspect, the plate insertion apparatus further includes a driver positioned within the cavity, the driver being biased by a spring and extending along the second axis. The plate insertion apparatus further includes a stem adjacent to the proximal end of the shaft, the stem extending from the shaft transverse to the first axis. The stem can include a threaded surface configured to be received by a grip attachment. The shaft can include a slot along the first axis. The shaft can be an external shaft, and the plate inserter includes an internal shaft positioned within the external shaft. The distal end of the external shaft can include an opening extending into the external shaft along the first axis, the opening communicating with the internal shaft. The internal shaft can be caused to be rotated via the opening such that a driver positioned within the cavity of the distal tip is rotated synchronously with the internal shaft. Rotation of the internal shaft in a first direction can cause a fastener received by the driver to be rotated in a fastening direction and rotation of the internal shaft in a second direction causes the fastener received by the driver to be rotated in an unfastening direction.

In accordance with another aspect of the present disclosure, a plate insertion apparatus includes a shaft, a distal tip, a linkage mechanism, and an actuator. The shaft extends along a first axis and has a proximal end and a distal end. The distal tip extends from the distal end of the shaft and includes a first claw, a second claw, and an opening between the first and second claws. The opening extends along a second axis. The linkage mechanism is coupled with the first and second claws. The actuator is adjacent to the proximal end of the shaft, wherein actuation of the actuator in a first direction causes movement of the linkage mechanism to expand the first and second claws and actuation of the actuator in a second direction causes movement of the linkage mechanism to contract the first and second claws.

In other embodiments of this aspect, the plate insertion apparatus further includes a stem adjacent to the proximal end of the shaft, the stem extending from the shaft transverse to the first axis. The linkage mechanism can be a four-bar linkage.

In accordance with another aspect of the present disclosure, a method for a surgical procedure utilizing a plate inserter includes: actuating an actuator of a plate inserter in a first direction to move a first claw away from a second claw; placing a plate and fastener between the first claw and the second claw; actuating the actuator in a second direction to clamp the plate between the first claw and the second claw; and fixing the plate to an interbody device with the fastener.

In other embodiments of this aspect, the plate inserter includes a first portion extending along a first axis and a second portion extending along a second axis, the first and second axes being transverse, the first and second claws included in the second portion. The plate inserter can further include an external shaft and an internal shaft connected with the fastener. The method can further include biasing the fastener towards the plate. The method can further include releasing the plate from the plate inserter by actuating the actuator in the first direction.

### BRIEF DESCRIPTION OF THE DRAWINGS

A more complete appreciation of the subject matter of the present invention and of the various advantages thereof can be realized by reference to the following detailed description in which reference is made to the accompanying drawings in which:

FIG. 1A is a top view of a plate inserter according to an embodiment of the present disclosure.

FIG. 1B is another top view of the plate inserter of FIG. 1A with a plate attached.

FIG. 1C is another top view of the plate inserter of FIG. 1A with the plate and a fastening member attached.

FIG. 1D is another top view of the plate inserter of FIG. 1A with the plate, fastening member, and an interbody attached.

FIG. 2A is a perspective view of the plate inserter of FIG. 1A.

FIG. 2B is another perspective view of the plate inserter of FIG. 1A with the plate attached

FIG. 2C is another perspective view of the plate inserter of FIG. 1A with the plate and fastening member attached

FIG. 2D is another perspective view of the plate inserter of FIG. 1A with the plate, fastening member, and interbody attached.

FIG. 2E is another perspective view of the plate inserter of FIG. 1A with the fastening member attached.

FIG. 3A is a cross-sectional view of the plate inserter of FIG. 1A taken along line 3-3 of FIG. 2A.

FIG. 3B is another cross-sectional view of the plate inserter of FIG. 1A taken along line 3-3 of FIG. 2A with the plate attached.

FIG. 3C is another cross-sectional view of the plate inserter of FIG. 1A taken along line 3-3 of FIG. 2A with the plate and fastening member attached.

FIG. 3D is another cross-sectional view of the plate inserter of FIG. 1A taken along line 3-3 of FIG. 2A with the plate, fastening member, and interbody attached.

FIG. 4A is an enlarged view of the distal end of the plate inserter of FIG. 3A.

FIG. 4B is another enlarged view of the distal end of the plate inserter of FIG. 3A with the plate attached.

FIG. 4C is another enlarged view of the distal end of the plate inserter of FIG. 3A with the plate and fastening member attached.

FIG. 4D is another enlarged view of the distal end of the plate inserter of FIG. 3A with the plate, fastening member, and interbody attached.

FIG. 5 is an enlarged perspective view of the distal end of the plate inserter of FIG. 1A.

FIG. 6A is a top view of a plate inserter according to another embodiment of the present disclosure.

FIG. 6B is another top view of the plate inserter of FIG. 6A with a plate attached.

FIG. 6C is another top view of the plate inserter of FIG. 6A with the plate and a fastening member attached.

FIG. 6D is another top view of the plate inserter of FIG. 6A with the plate, fastening member, and an interbody attached.

FIG. 7 is a perspective view of the plate inserter of FIG. 6A.

FIG. 8 is a cross-sectional view of the plate inserter of FIG. 6A taken along line 7-7 of FIG. 7.

FIG. 9 is an enlarged view of the distal end of the plate inserter of FIG. 8.

FIG. 10 is an enlarged perspective view of the distal end of the plate inserter of FIG. 6A.

FIG. 11 is a flowchart illustrating a method for utilizing a plate inserter according to an embodiment of the present disclosure.

FIG. 12A is a side view of a slap hammer according to another embodiment of the present disclosure.

FIG. 12B is a perspective view of the slap hammer of FIG. 12A.

### DETAILED DESCRIPTION

In describing the preferred embodiments of the inventions, specific terminology will be used for the sake of clarity. However, the inventions are not intended to be limited to any specific terms used herein, and it is to be understood that each specific term includes all technical equivalents, which operate in a similar manner to accomplish a similar purpose. In the drawings and in the description which follows, the term “proximal” refers to the end of instrument, or a portion thereof, which is closest to the operator in use, while the term “distal” refers to the end of the instrument, or portion thereof, which is farthest from the operator in use. When referring to the human body, the term “proximal” means closer to the heart, the term “distal” means more distant form the heart, the term “anterior” means towards the front part of the body or the face and the term “posterior” means towards the back of the body. The term “medial” means toward the midline of the body and the term “lateral” means away from the midline of the body.

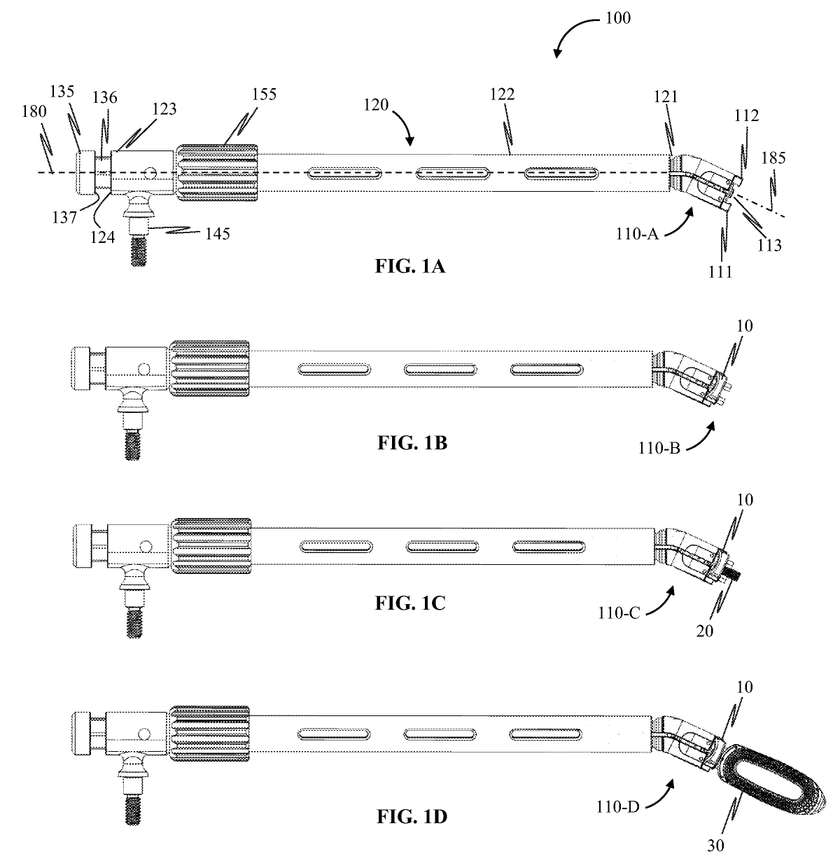

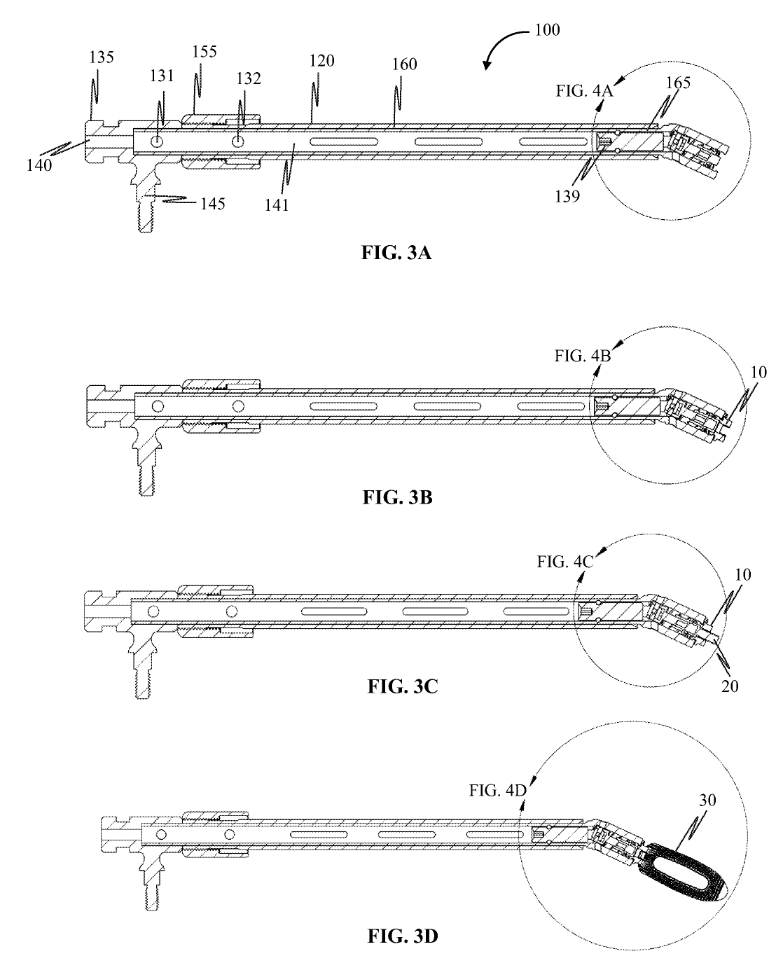

FIG. 1A depicts one embodiment of a plate inserter 100 according to the present disclosure. Plate inserter 100 includes a distal tip 110, a housing 120, an actuator 155, and a grip adapter 145. Housing 120 includes a proximal end 123, a distal end 121, and a length 122 extending along a first axis 180. Housing 120 is cannulated and configured to house an inner shaft (such as inner shaft 160 shown in FIG. 3).

Distal tip 110 extends from distal end 121 of housing 120 along a second axis 185, which is different from first axis 180. Distal tip 110 includes a first claw 111, a second claw 112, a space between first and second claws 111-112, and a driver 113 positioned within the space. Claws 111-112 are adjusted by actuator 155. Actuator 155 is adjacent to proximal end 123 of housing 120. Actuation of actuator 155 in a first direction causes first and second claws 111-112 to expand such that the distance between first claw 111 and second claw 112 increases. Actuation of actuator 155 in a second direction causes first and second claws 111-112 to contract such that the distance between first claw 111 and second claw 112 decreases. Claws 111-112 of distal tip 110 are configured to receive the plate. Particularly, claws 111-112 are configured to clamp onto the plate that is configured to be attached to at least one vertebral body when actuator 155 is actuated in the first direction. Moreover, claws 111-112 are configured to release the inserted plate by actuation of actuator 155 in the second direction. Claws 111-112 each have a lip such that the plate can be secured to plate inserter 100.

Driver 113 is biased by a spring and extends along second axis 185. Driver 113 is configured to receive the fastener, which is configured to fasten the plate with the interbody. In an embodiment with the plate and fastening member inserted onto plate inserter 100, the spring of driver 113 biases the fastening member against the plate such that the fastening member remains coupled with the plate. As distal tip 110 advances, it is configured to drive (e.g., rotate) the fastening member. In some embodiments, driver 113 has a hexalobe shaped end configured to receive a hexalobe compatible fastener.

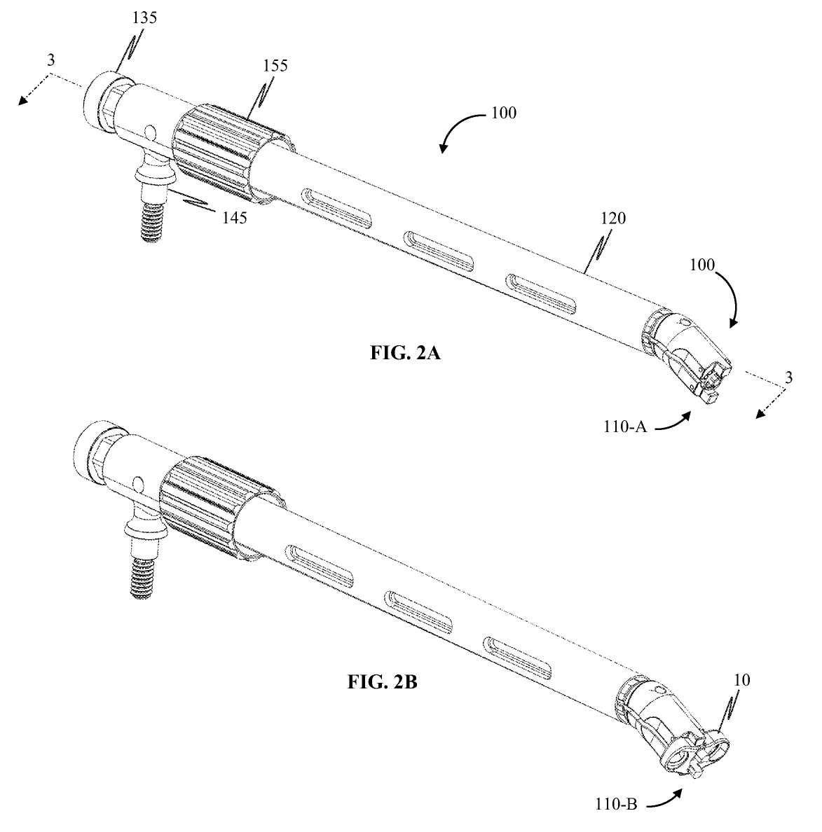

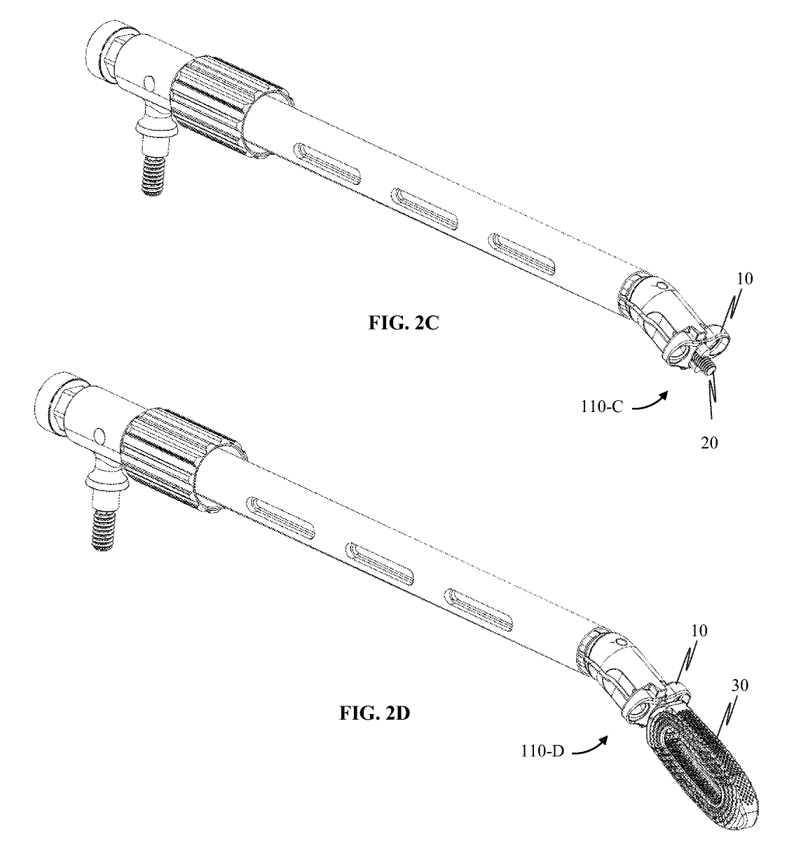

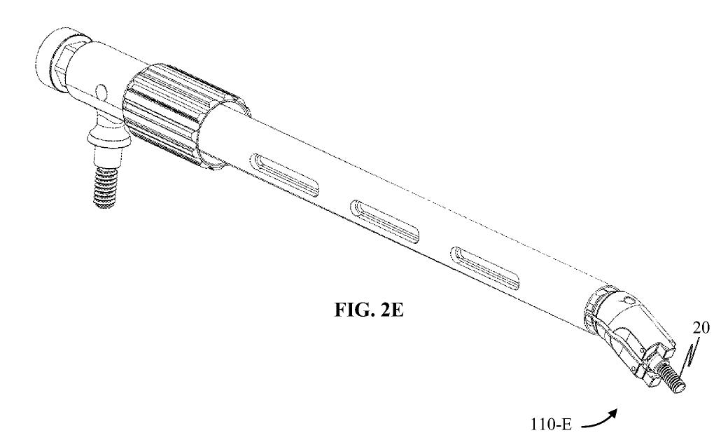

As shown in e.g., FIGS. 1A-1D, distal tip 110 has multiple configurations. Distal tip 110-A is shown to have a plate inserter with no attachments thereon. However, plate inserter 100 may have a plate 10, a fastening member 20, an interbody 30, and any combination thereof coupled with distal tip 110 thereon. FIG. 1B shows plate inserter 100 with plate 10 coupled with claws 111-112 at distal tip 110-B. FIG. 1C shows distal tip 100-C wherein plate 10 is coupled with claws 111-112 and fastening member 20 is coupled with plate 10 and driver 113. FIG. 1D shows distal tip 110-D wherein all three of plate 10, fastening member 20, and interbody 30 are coupled with plate inserter 100. It should be appreciated that other configurations are possible such as shown in FIG. 2E where distal tip 100-E includes fastening member 20 coupled with driver 113.

In an embodiment where the plate and fastening member are attached to plate inserter 100, they can be inserted into a patient simultaneously and coupled with an already inserted interbody. In another embodiment, the interbody is coupled with the plate and the fastening member on distal end 110 such that all three components are inserted simultaneously. Simultaneous insertion of either the plate and fastening member or plate, fastening member, and interbody is advantageous at least because it minimizes the amount of entries necessary for a successful surgical operation and the amount of time needed for the operation. Less surgical entries with instruments as well as shorter operation times lead to increased operation success.

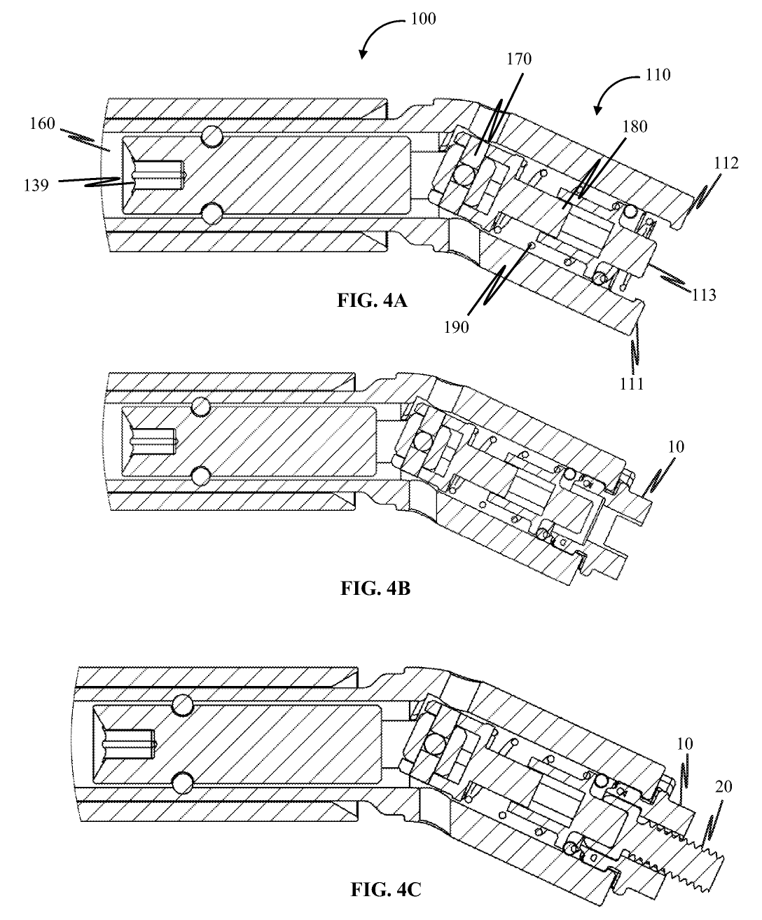

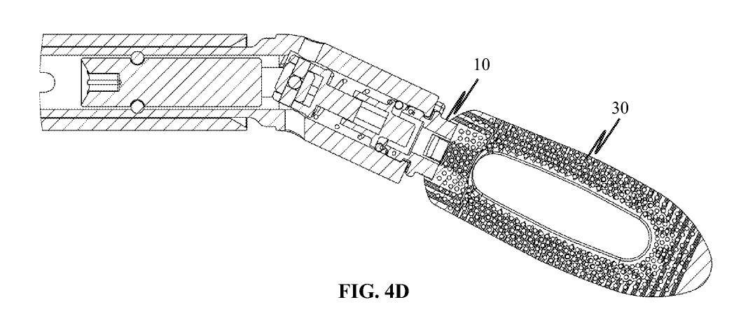

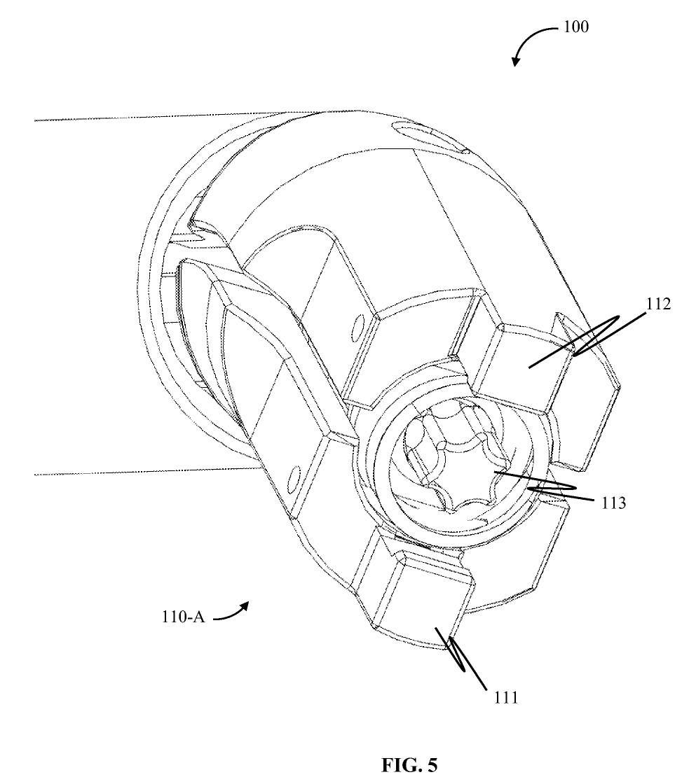

Claws 111-112 are actuated by actuator 155 positioned adjacent to proximal end 123. As shown in connection with FIGS. 3-4, actuator 155 includes an interior surface having a plurality of first engagement members configured to engage with a plurality of second engagement members of an outer surface of housing 120. The engagement members comprise of threaded surfaces but can comprise of any other surface known in the art. The threading facilitates actuation of claws 111-112 by actuating shaft 120. Specifically, actuation in the first direction causes shaft 120 to move in a proximal-to-distal direction such that shaft 120 advances relative to internal shaft 160. The advancement of shaft 120 causes a living hinge connecting claws 111-112 to be compressed towards each other as distal end of shaft 120 advances relative to shaft 160. Actuation in a second direction, opposite the first direction, causes shaft 120 to move in a distal-to-proximal direction such that shaft 120 retreats relative to internal shaft 160. The movement of shaft 120 causes the living hinge to be decompressed such that claws 111-112 are caused to move away from one another.

Grip adapter 145 is adjacent to proximal end 123 of housing 120 and extends transversely from first axis 180. As shown, grip adapter 145 includes a threaded surface such that it can receive a component with matching thread. In an embodiment, the component received by grip adapter 145 includes a handle (not shown) configured to be held by an operator of plate inserter 100 such as during an operation.

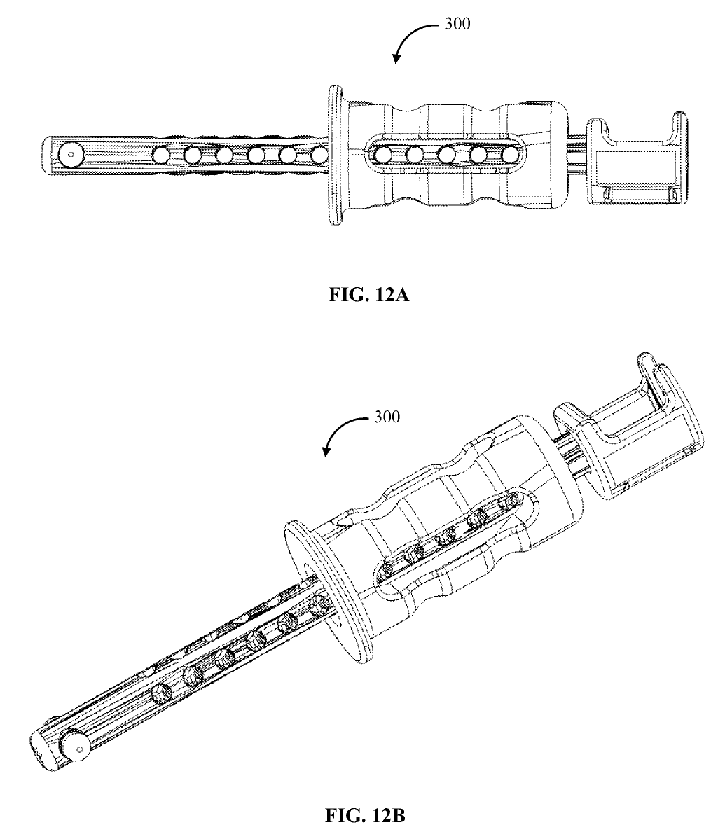

Knob 135 extends from an opening of housing 120 at proximal end 123. Knob 135 includes a groove 136 and a distal face 137 facing a proximal face 124 of housing 120 adjacent to proximal end 123. Knob 135 is communicatively connected with an inner shaft of housing 120 such as inner shaft 160 as shown in FIG. 3. Knob 135 and groove 136 are slap hammer attachments configured to be received and actuated by any slap hammer known in the art such as e.g., slap hammer 300 as shown in FIGS. 12A-B.

Plate inserter 100 further includes a first bore 140 and a second bore 141. First bore 140 extends from a proximal face of knob 135 terminating within plate inserter 100. Second bore 141 extends from a distal end of first bore to an engagement member 139 near distal tip 110. First bore 140 and second bore 141 communicate with inner shaft 160. Inner shaft 160 has a longitudinal length that extends along first axis 180 and is configured to be encompassed at least substantially within housing 120. Inner shaft 160 is substantially hollow and encompasses second bore 141.

A driver is configured to be inserted at an opening of first bore 140 from the proximal face of knob 135 such that the driver can extend through first bore 140 and second bore 141 to engagement member 139. The driver is configured to engage with engagement member 139 such that a distal shaft 165 is caused to be actuated. Particularly, rotation of the driver while engaged with engagement member 139 causes distal shaft 165 to rotate which causes driver 113 to rotate via a coupler 170 coupled with distal shaft 165 and driver 113. Moreover, rotation of the driver is configured to rotate distal shaft 165 within housing 120 as opposed to rotating housing 120 itself.

Plate inserter 100 further includes a first pin 131 and a second pin 132. First pin 131 is coupled with inner shaft 160 and shaft 120 and is configured to secure inner shaft 160 with bore 140. Second pin 132 is coupled with inner shaft 160 and actuator 155 and is configured to impede actuator 155 from translating relative to inner shaft 160.

As shown in FIG. 4A, inner shaft 160 is coupled with a distal tip shaft 180 via a coupler 170. Coupler 170 is configured to translate the rotation of distal shaft 165 to distal tip shaft 180. Coupler 170 can be but is not limited to a universal joint. Distal tip shaft 180 is coupled with driver 113 such that rotation of distal tip shaft 180 translates to driver 113 causing driver 113 to rotate. Driver 113 is configured to receive a fastening member thereon such that as driver 113 is caused to be rotated the fastening member is also rotated. The fastening member can include but is not limited to, a screw, a bolt, a rivet, a nail, the like, and any combination thereof. Distal tip 110 further includes compression member 190 coupled with distal tip shaft 180 and driver 113. Compression member 190 is configured to apply a force along the second axis towards an opening of distal tip 110 such that a fastening member attached to driver 113 has a force applied towards the opening of distal tip 110. In an embodiment, compression member 190 is in a compressed state when claws 111-112 have a plate clamped thereon such that compression member 190 has potential energy. As a non-limiting example, the free length of compression member 190 extends beyond the opening of distal tip 110 such that when a plate is inserted compression member 190 is loaded and causes a fastening member inserted onto the plate to be pushed towards the opening. This is desirable as both the insertion of a plate into a patient and the attachment of the plate to an interbody can be achieved with plate inserter 100 at the same time. Moreover, the angled tip of plate inserter 100 allows for an ATP/OLIF approach otherwise not achievable with other plate inserters known in the art. It should be appreciated that plate inserter 100 can be utilized in other approaches other than an ATP/OLIF approach such as, but not limited to, ALIF, XLIF, TLIF, PLIF, and any other approach known in the art. Furthermore, compression member 190 can be caused to depress the inserted fastening member within distal tip 110 such that compression member 190 is not continuously pushing the fastening member forward. Compression member 190 can be further actuated such that compression member 190 biases the fastening member against the inserted plate. As a non-limiting example, an operator may actuate plate inserter 100 causing fastening member to be depressed against compression member 190, then insert plate inserter 100 into a patient, followed by actuating plate inserter 100 again to push the fastening member forward when aligned with an inserted interbody.

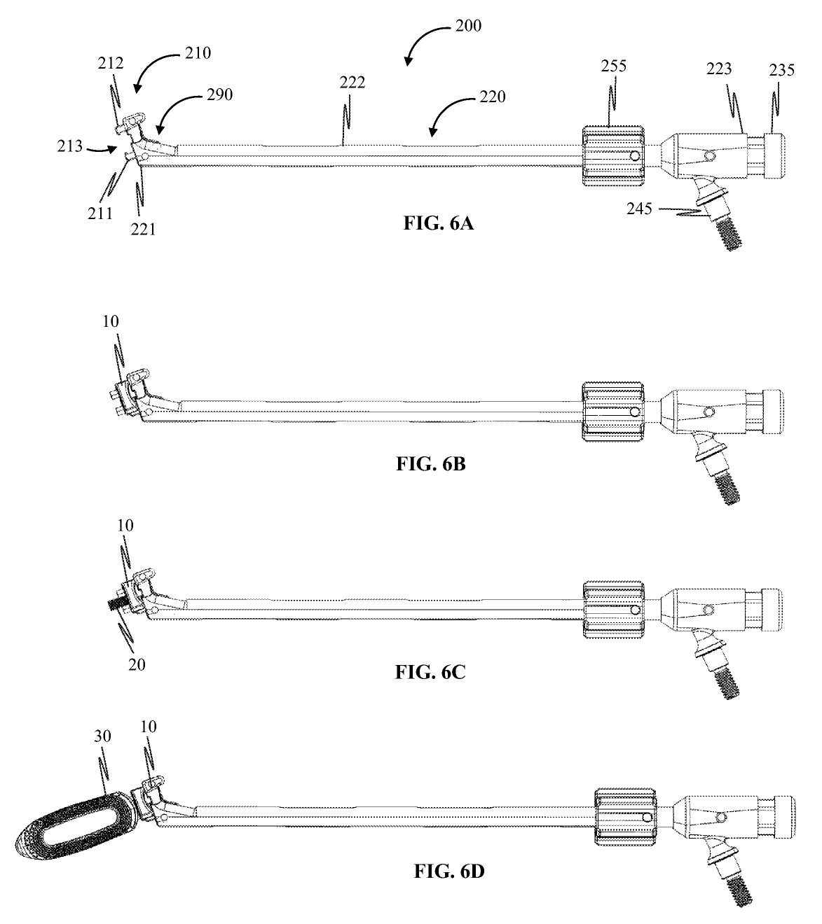

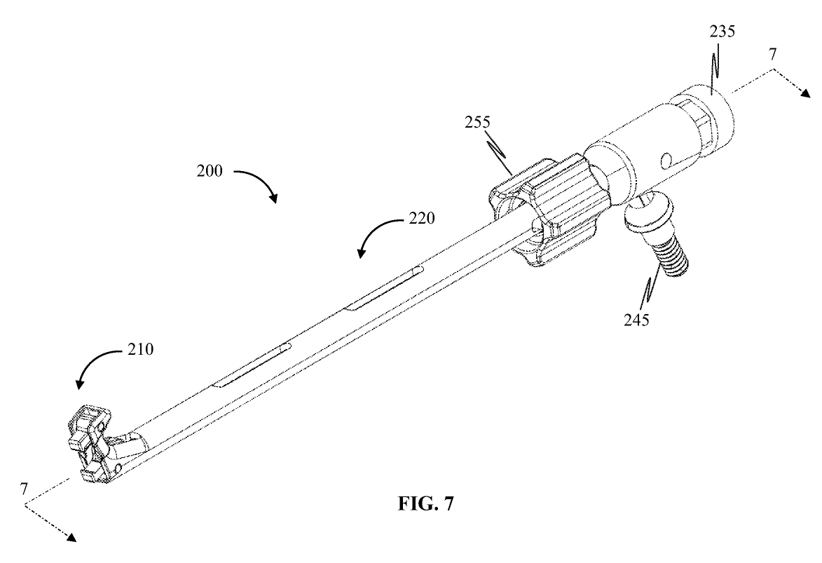

FIG. 6A depicts another embodiment of a plate inserter 200 of the present disclosure. Plate inserter 200 is similar to plate inserter 100, and therefore like elements referred to with similar numerals within 200-series of numbers. For example, plate inserter 200 includes a distal tip 210, a housing 220, a grip adapter 245, and an actuator 255. However, unlike plate inserter 100, plate inserter 200 does not include a driver configured to receive a fastener. Specifically, distal tip 210 of plate inserter 200 is configured to receive and insert a plate 10 coupled with a fastening member 20 coupled with an interbody 30.

Housing 220 of plate inserter 200 includes a body 222 extending along a first axis and has a proximal end 223 and a distal end 221. Housing 220 is configured to house an inner shaft 260.

Distal tip 210 is coupled with housing 220 and extends from distal end 221 along a second axis different from the first axis. Distal tip 210 includes a first claw 211, a second claw 212, and a space 213 between first and second claws 211-212. Claws 211-212 are configured to receive and clamp onto a plate. The distance between claws 211-212 is configured to increase or decrease such that the plate may be secured or released. Distal tip 210 further includes a linkage mechanism 290 configured to be adjusted to change the distance between claws 211-212. Linkage mechanism 290 is adjusted by actuator 255.

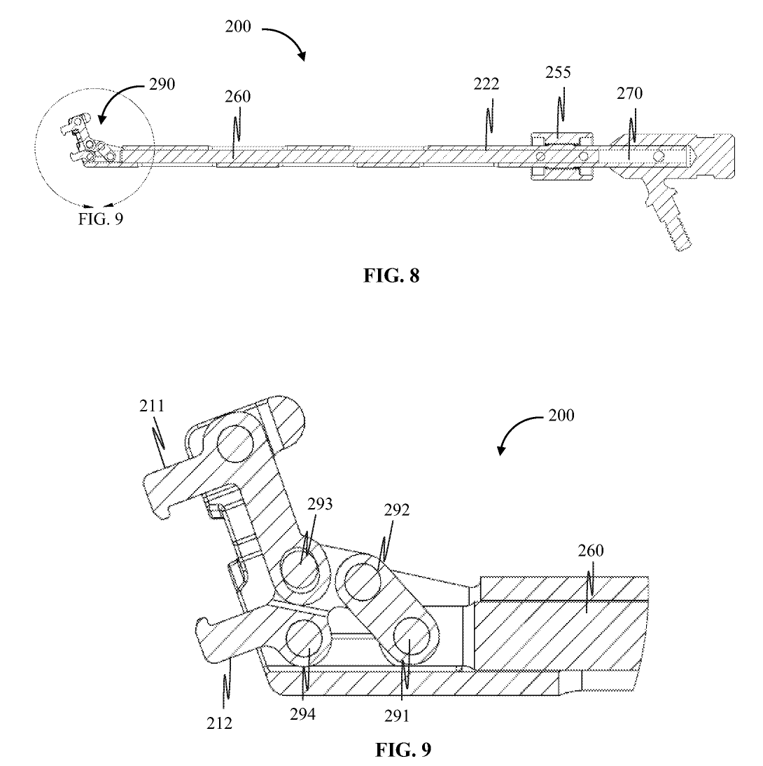

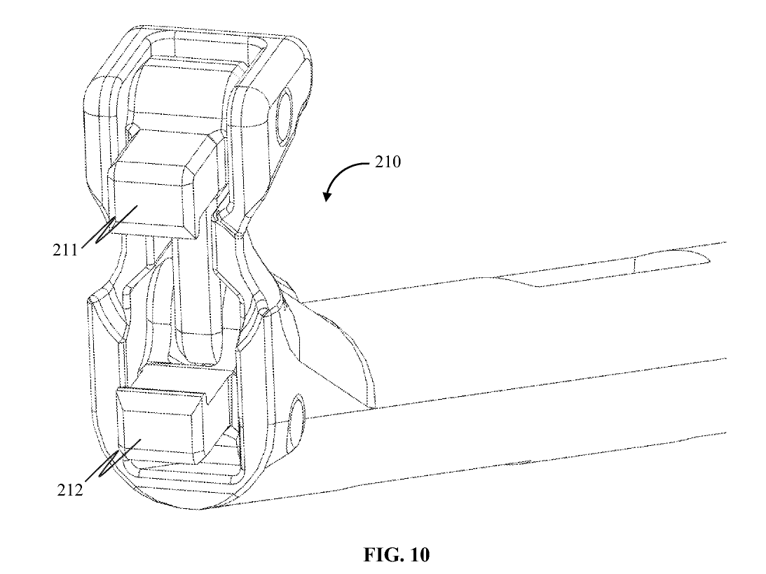

Actuator 255 is coupled with body 222 of housing 220. As shown in FIG. 8, actuator 255 includes an internal surface with engagement members configured to engage with engagement members of an external surface of body 222. In an embodiment, the engagement members are threaded surfaces such that actuator 255 can be rotated similar to that of a threaded nut on a bolt. Movement of actuator 255 in a first direction causes linkage mechanism 290 to move in a first path which subsequently causes claws 211-212 to contract. Movement of actuator 255 in a second direction causes linkage mechanism 290 to move in a second path which subsequently causes claws 211-212 to expand.

Grip adapter 245 is adjacent to proximal end 223 of housing 220 and extends transversely from a longitudinal length of housing 220. As shown, grip adapter 245 is angled approximately at sixty degrees and includes a threaded surface such that it is configured to receive a component with a matching thread. In an embodiment, the component with matching thread received by grip adapter 245 includes a handle attachment configured to be held by an operator utilizing plate inserter 200 during operation.

Plate inserter 200 further includes knob 235. Knob 235 is a slap hammer attachment and is configured to be received by any slap hammer known in the art such as e.g., slap hammer 300 as shown in FIGS. 12A-B.

FIG. 8 is a cross-sectional view of plate inserter 200 of FIG. 6A. As shown, inner shaft 260 is encompassed by housing 220. Inner shaft 260 is configured to translate along the first axis, the same axis as the longitudinal length of housing 220. Actuator 255 is configured to be actuated in either a first or second direction causing inner shaft 260 to translate either in a proximal or distal direction. As shown, actuator 255 and a portion of an outer surface of body 222 each have a plurality of engagement members configured to be received by one another. Actuation of actuator 255 in a first direction causes inner shaft 260 to translate distally towards distal end 221. Actuation of actuator 255 in a second direction causes inner shaft 260 to translate proximally towards proximal end 223.

A linkage mechanism 290 is coupled with claws 211-212 and inner shaft 260 such that translation of inner shaft 260 causes linkage mechanism 290 to move claws 211-212. Specifically, translation of inner shaft 260 proximally towards a space 270 causes a linkage of linkage mechanism 290 to also partially move proximally such that the distance between first claw 211 and second claw 212 narrows. Translation of inner shaft 260 distally towards distal tip 210 also causes a linkage of linkage mechanism 290 to move distally such that the distance between first and second claws 211-212 widens.

As shown in FIG. 9, linkage mechanism 290 is a four-bar linkage and includes four links: first link 291, second link 292, third link 293, and fourth link 294. When inner shaft 260 is translated proximally, first link 291 moves distally such that the angle between first link 291 and second link 292 becomes closer to that of the first axis. Subsequently, the downward movement of second link 292 causes fourth link 294 to pivot causing second claw 212 to move closer to first claw 211. Furthermore, the pivot causes third link 293 to move proximally such that first claw 211 moves towards second claw 212. Translation of inner shaft 260 distally inverts this process such that the distance between first claw 211 and second claw 212 increases.

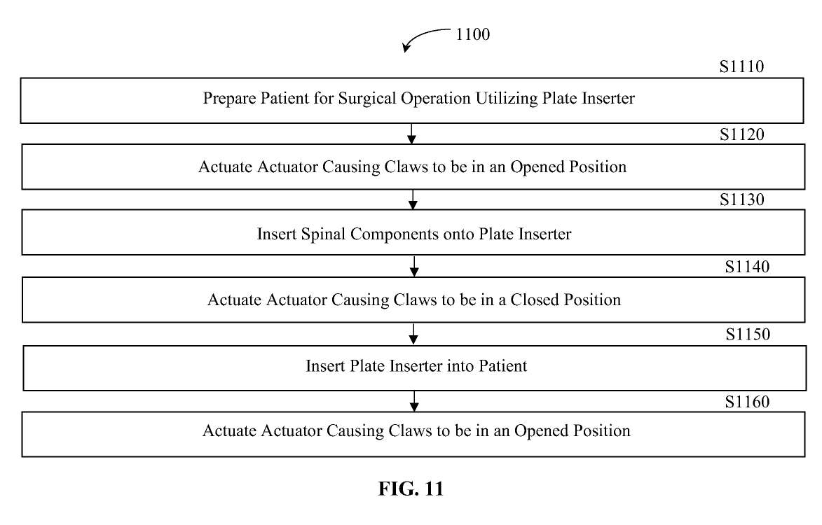

FIG. 11 is a flowchart illustrating a method for utilizing a plate inserter according to an embodiment of the present disclosure. In an embodiment, the method may be performed by plate inserter 100 or plate inserter 200.

At S1110, a patient is prepared for a surgical operation utilizing a plate inserter. Preparation of a patient for operation includes any and all procedures known in the art. These can include anesthetizing the patient, positioning the patient on the operating table, sterilizing the patient and instruments, performing any other known pre-operation procedures, a combination thereof, and the like.

At S1120, an actuator of the plate inserter is actuated causing claws of the plate inserter to be in an opened position. Actuation of the actuator in a first direction causes a distance between a first claw and a second claw to increase. The expansion between the claws makes the plate inserter receivable by spinal components such as a plate, an interbody device, and a fastening member configured to join the plate and the interbody device. In an embodiment, the actuator is either actuator 155 or actuator 255.

At S1130, the spinal components are inserted onto the plate inserter. Specifically, the claws of the plate inserter are configured to receive the edges of the plate wherein the fastening member and or interbody device can be coupled with the plate. In an embodiment, the fastening member can be inserted onto a driver of the plate inserter between the first and second claws.

At S1140, the actuator of the plate inserter is actuated causing claws of the plate inserter to be in a closed position. Actuation of the actuator in a second direction is configured to cause the distance between the first claw and the second claw to decrease. Accordingly, the inserted plate is clamped by the first and second claws of the plate inserter. Any other components such as the fastening member and the interbody device can also be attached.

At S1150, the plate inserter is inserted into the patient. In an embodiment where the plate and fastening member are attached, the plate inserter is configured to drive the fastening member forward as the plate inserter advances such that the fastening member can fasten with the interbody device attaching the interbody device with the plate. As a non-limiting example, the interbody device can be implanted into the patient and subsequently the plate inserter with the plate and fastening member attached thereon can be inserted into the patient and ultimately attached to the interbody device. In such example, the fastening member can be actuated utilizing a driver of the plate inserter. In another non-limiting example, the interbody device can be attached with the plate and fastening member such that the plate inserter simply inserts all three components into the designated area.

At S1160, the actuator of the plate inserter is actuated causing claws of the plate inserter to be in an opened position. Once the interbody device is appropriately positioned within the spine and is attached to the plate, the actuator can be actuated causing the claws to release the plate. Thereafter, the plate inserter can be retracted from the patient leaving the plate, fastening member, and interbody device in the patient. The surgical operation can then be wound up such as by suturing the incision and any other procedure necessary to complete the operation.

It is to be understood that the various components discussed herein can be constructed of any suitable material for use in the human body. For instance, it is contemplated to construct the various components of metallic materials, such as titanium and stainless steel. It is also contemplated to use various manufacturing methods to construct the components, including additive manufacturing.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A plate insertion apparatus comprising:
a shaft having a proximal end and a distal end, the shaft extending along a first axis;
a distal tip extending from the distal end along a second axis, the distal tip including a first claw, a second claw, and a cavity between the first and second claws, the second axis being different from the first axis; and
an actuator adjacent the proximal end, wherein actuation of the actuator in a first direction causes the first and second claws to expand and actuation of the actuator in a second direction causes the first and second claws to contract.

2. The plate insertion apparatus of claim 1, wherein the plate insertion apparatus further includes a driver positioned within the cavity, the driver being biased by a spring and extending along the second axis.

3. The plate insertion apparatus of claim 1, wherein the distal tip includes a hexalobe shaped end configured to receive a hexalobe shaped fastener.

4. The plate insertion apparatus of claim 1, wherein the plate insertion apparatus further includes a stem adjacent to the proximal end of the shaft, the stem extending from the shaft transverse to the first axis.

5. The plate insertion apparatus of claim 4, wherein the stem includes a threaded surface configured to be received by a grip attachment.

6. The plate insertion apparatus of claim 1, wherein the shaft includes a slot along the first axis.

7. The plate insertion apparatus of claim 1, wherein the shaft is an external shaft, and the plate inserter includes an internal shaft positioned within the external shaft.

8. The plate insertion apparatus of claim 7, wherein the distal end of the external shaft includes an opening extending into the external shaft along the first axis, the opening communicating with the internal shaft.

9. The plate insertion apparatus of claim 8, wherein the internal shaft is caused to be rotated via the opening such that a driver positioned within the cavity of the distal tip is rotated synchronously with the internal shaft.

10. The plate insertion apparatus of claim 9, wherein rotation of the internal shaft in a first direction causes a fastener received by the driver to be rotated in a fastening direction and rotation of the internal shaft in a second direction causes the fastener received by the driver to be rotated in an unfastening direction.

11. A plate insertion apparatus comprising:
a shaft having a proximal end and a distal end, the shaft extending along a first axis;
a distal tip extending from the distal end, the distal tip including a first claw a second claw, and an opening between the first and second claws, the opening extending along a second axis;
a linkage mechanism coupled with the first and second claws; and
an actuator adjacent the proximal end, wherein actuation of the actuator in a first direction causes movement of the linkage mechanism to expand the first and second claws and actuation of the actuator in a second direction causes movement of the linkage mechanism to contract the first and second claws.

12. The plate insertion apparatus of claim 11, wherein the plate insertion apparatus further includes a stem adjacent to the proximal end of the shaft, the stem extending from the shaft transverse to the first axis.

13. The plate insertion apparatus of claim 11, wherein the linkage mechanism is a four-bar linkage.

14. A method for a surgical procedure utilizing a plate inserter, the method comprising:
actuating an actuator of a plate inserter in a first direction to move a first claw away from a second claw;
placing a plate and fastener between the first claw and the second claw;
actuating the actuator in a second direction to clamp the plate between the first claw and the second claw; and
fixing the plate to an interbody device with the fastener.

15. The method of claim 14, wherein the plate inserter comprises:
a first portion extending along a first axis and a second portion extending along a second axis, the first and second axes being transverse, the first and second claws included in the second portion.

16. The method of claim 14, wherein the plate inserter includes an external shaft and an internal shaft connected with the fastener.

17. The method of claim 16, the method further comprising:
causing the internal shaft to be rotated, wherein rotation of the internal shaft in a first direction causes the fastener to thread into the interbody and rotation of the internal shaft in a second direction causes the fastener to unthread from the interbody.

18. The method of claim 14, the method further comprising biasing the fastener towards the plate.

19. The method of claim 14, the method further comprising:
releasing the plate from the plate inserter by actuating the actuator in the first direction.
